(19) United States
(12) Patent Application Publication
(10) Pub. No.: US 2022/0202784 A1
(43) Pub. Date: Jun. 30, 2022

(54) METHODS AND COMPOSITIONS FOR INCREASING THE EFFECTIVENESS OF ANTIVIRAL AGENTS

(71) Applicant: Antivirus Therapeutics, Princeton Junction, NJ (US)

(72) Inventors: Anand Gumaste, Princeton Junction, NJ (US); David A. Byron, Lawrenceville, NJ (US)

(21) Appl. No.: 17/697,298

(22) Filed: Mar. 17, 2022

Related U.S. Application Data
(63) Continuation of application No. 17/016,200, filed on Sep. 9, 2020, now abandoned.
Application No. 17/016,200 is a continuation of application No. 16/749,209, filed on Jan. 22, 2020, now abandoned.
Application No. 16/749,209 is a continuation of application No. 16/432,185, filed on Jun. 5, 2019, now abandoned.
Application No. 16/432,185 is a continuation of application No. 14/911,465, filed on Feb. 10, 2016, now abandoned.
(60) Provisional application No. 61/866,090, filed on Aug. 15, 2013.

(51) Int. Cl. A61K 31/4245; A61K 9/00; A61K 47/69; A61K 9/16

(52) U.S. Cl. CPC A61K 31/4245; A61K 9/0043; A61K 9/1652; A61K 9/0053; A61K 47/6951

(57) ABSTRACT

Compositions and method of the present invention comprise novel formulations for increasing the effectiveness of antiviral agents and for preventing and treating symptoms associated with the common cold and viral infections. The present invention is directed to treating symptoms resulting from viral infections and diseases associated with Picomaviridae, Coronaviridae, Orthomyxoviridae, Paramyxovirinae, Reoviridae, and Adenoviridae. The novel formulations provided herein improve the therapeutic ratio of antiviral agents such as pleconaril.

### FIELD OF THE INVENTION

The present invention relates to compounds, compositions and methods for increasing the effectiveness of antiviral agents and for preventing and treating respiratory ailments. The present invention further provides improved therapeutics for the prevention and treatment of acute viral respiratory infection, such as the common cold, in adults and children. The present invention relates generally to novel compositions and methods for the treatment of acute picornaviral respiratory infection together with subsets of this disease condition. More specifically, the present invention comprises novel formulations for improving the efficacy of antiviral agents such as pleconaril.

### BACKGROUND OF THE INVENTION

Acute picornaviral respiratory illness (common cold) in adults and children, the most prevalent contagious viral disease in humans, still lacks a safe and effective antiviral treatment. The Picornaviridae family (picornaviruses) comprises the principle viral pathogens associated with the common cold and upper respiratory tract infection (URI). Two genera of picornavirdae, the rhinoviruses and enteroviruses, are responsible for greater than 0% of episodes of nom-influenza-virus respiratory infection annually, and rhinoviruses account for 60% to SO % of all cold episodes. Rhinorrhea, sore threat, cough and headache are among the main symptoms of the infection and URI can cause frequent asthma and chronic obstructive pulmonary disorder (COPD) exacerbations. There is no treatment currently available for the viral (picornaviral) respiratory infection (common cold) and there is limited data on the efficacy of cold symptom relief medication.

Picornavirus is transmitted mainly from contact with the saliva or nasal secretions of an infected person, either directly, in aerosol form generated by coughing and sneezing, or from contaminated surfaces. Symptoms are not necessary for viral shedding or transmission, as a percentage of asymptomatic subjects' exhibit viruses in nasal swabs. It is not always possible to identify the virus type through symptoms, although influenza can be distinguished by its sudden onset, fever, and cough: however, physicians properly diagnose the common cold with a high degree of accuracy. Furthermore, adults are quite good at predicting the early onset of a picornavirus infection. Rhinovirus colds do not generally cause damage to the nasal epithelium. Macrophages trigger the production of cytokines, which in combination with mediators cause the symptoms. Cytokines cause the systemic effects. The mediator bradykinin plays a major role in causing the local symptoms such as sore throat and nasal irritation. Symptoms usually begin 2 to 5 days after initial infection but occasionally occur in as little as 10 hours after exposure. Rhinorrhea, sore throat, cough and headache are sometimes accompanied by muscle aches, fatigue, malaise, weakness, or loss of appetite.

Acute picornaviral upper respiratory infection (common cold) is prevalent worldwide with significant associated morbidity. For example, is estimated that every year, Americans get approximately 1 billion colds, with roughly 60 million Americans being affected with three to five colds. Statistics show that preschool-aged children have around nine colds per year, kindergartners can have approximately 12 colds per year, and adolescents and adults have about seven colds per year. Cold season runs from September until March or April, so children usually catch most cold viruses during these months. Children are two to three times more likely than adults to get sick with the flu, and children frequently spread the virus to others. Although an alarming 22 million school days are lost every year due to the common cold, most cold medications are not intended to be taken by children. The directions for a common cold remedy will typically say “not for children under 12” and may recommend doctor consultation. Additionally, parents are advised not to give children aspirin or products that contain aspirin because of the established link between aspirin use, viral infections and Reye's syndrome, a rare, but sometimes life threatening disease than can follow viral infections in children. A number of infant and toddler deaths have been associated with overdoses of over the counter cold remedies.

In a study conducted by Bramley et al. in 2002 (J. Occup Environ Med. 2002 Sept; 44(9):822-9), it was concluded that each cold experienced by a working adult in the US caused an average of 8.7 lost work hours (2.8 absenteeism hours; 5.9 hours of on-the-job loss), and 1.2 work hours were lost because of attending to children under the age of 13 who were suffering from colds. Bramley et al. concluded that the economic cost of lost productivity due to the common cold approaches $25 billion, of which $16.6 billion is attributed to on-the-job productivity loss, $8 billion is attributed to absenteeism, and $230 million is attributed to caregiver absenteeism. Other reports indicate that in the United States, the common cold leads to 75 to 100 million physician visits annually at a conservative cost estimate of $7.7 billion per year. Americans spend $2.9 billion on over-the-counter drugs and another $400 million on prescription medicines for symptomatic relief.

Viral infections are especially dangerous in vulnerable populations such as preterm newborn infants, frail elderly and immunocompromised. In severe cases such as preterm infants or children suffering from cystic fibrosis, infection with cold-associated viruses such as RSV, Coxsackic virus B, and the results arc fatal.

Many therapeutics target particular stages in the life cycle of a virus. For example, certain therapeutics target the binding of the virus to a host cell surface, whereas others target replication or protein synthesis. Others target uncoating of virus (loss of protein coat, fusion of lipid membrane with endosome/lysosome), uptake into intracellular vesicles (endosomes) transcription of genome to new RNA or DNA (polymerases are the target), integration of the viral DNA into chromosomal DNA of the host cell (where this occurs), mRNA transcription, mRNA processing (polyadenylation, methylation, capping, splicing), translation to protein, post-translational modification of proteins (glycosylation, phosphorylation, fatty acylation, proteolysis) or assembly of the components into the whole vims.

Despite the prevalence of “colds” and the significant negative impact caused by the illness, no adequately effective therapeutics are available. Physicians indicate that 35% of patients presenting a cold are prescribed a cough medication, and the numerous negative side-effects of cold and cough over the counter products is well documented: for example, the alpha-adrenergic activity of pseudoephedrine, responsible for its decongestant activity, cause cardiovascular and CNS problems and complications. Over the counter drugs such as antihistamines have been evaluated for their effectiveness in treating common cold symptoms and while research indicates that these products are safe when used as directed, many of them are ineffective. Seeking treatment for a common cold is one of the leading causes of doctor visits, though doctors can do little to treat the illness. Antibiotics are ineffective and are not taken for common cold prevention or treatment. Over use of antibiotics is believed to be the cause of an increase in more resistant strains of bacteria.

What is needed therefore is an effective drug development strategy for combating the numerous symptoms and complications associated with common cold type illnesses. Such a strategy may include the development of improved formulations having faster dissolution rates for higher local concentration of medication combined with a longer residence time allowing prolonged contact and enhancing the chances of a drug to penetrate past physiological barriers such as the turbinates, and effectively reach target areas such as the adenoids. What is also needed, are formulations combining the therapeutic effect of antiviral agents synergistically with the action of selected pharmaceutical excipients that may both enhance therapeutic effect and add desirable physicochemical properties to the formulations.

### SUMMARY OF THE INVENTION

Disclosed herein are improved methods and compositions for the treatment of symptoms and ailments associated with the common cold. (The word treatment is meant to encompass treatment and/or prophylaxis use of the invention.) Such symptoms include, but are not limited to rhinorrhea, runny nose, general congestion, nasal congestion, sneezing, fever, sore throat, cough, headache, body ache, muscle aches, muscle weakness, malaise, exhaustion, uncontrollable shivering, chills, otitis media, loss of appetite, pneumonia and bronchiolitis. In addition, the novel compositions and methods disclosed herein are particularly desirable due to improved stability, low dosing levels, ease of dosing and administration as well as the significant reduction and absence of side effects and toxicity. Surprisingly, the methods, compositions and formulations described herein display unexpected results with regard to therapeutic efficacy.

More specifically, the improved formulations and therapeutics taught herein are directed to novel strategies for improving the therapeutic ratio of antiviral agents. In certain embodiments, the compositions have increased efficacy as a result of targeted site delivery, control of API particle size, and formulations having synergistic effects. In addition, efficacy is improved by controlling dosing regimen and expanding the therapeutic window. The compositions of the present invention are further desirable as they have reduced toxicity and improved stability.

Disclosed herein arc unique and improved formulations of antiviral agents such as pleconaril. Pleconaril is an antiviral drug originally designed for oral delivery. As originally delivered, the drug was unsuccessful for its intended use (therapeutic effect on illness associated with the common cold) because of various factors including but not limited to drug interaction due to CYP 3A induction. The problems previously encountered have been overcome herein as a result of an improved formulation and therapeutic administration. The unique methods and compositions of the present invention include improvements including, but not limited to, optimized viscosities, pediatric appropriate formulations (volumes, dye-free antiviral suspensions, syrups), optimized nasal formulations (having unexpected sustained action, and improved formulation stability), unique methods of administration, and novel combinations with additional pharmaceutical agents for greater synergy.

Disclosed herein are novel antiviral agent formulations and methods of their preparation resulting in pharmaceutical products associated with treatment and prevention of different viral infections, in particular with treatment and prevention viral infections using respiratory drug delivery, wherein such formulations and compositions are optimized for ease of delivery, for stability and reduced toxicity.

Disclosed herein are novel compositions and methods for alleviating and preventing symptoms associated with the common cold, particularly those viral infections and diseases associated with Picornaviridae (i.e. Enterovirus, Hepatovirus, Rhinovirus), Coronaviridae (SARS virus), Orthomyxoviridae (Influenzavirus) Paramyxovirinae (Pneumovirinae, Paramyxovirus, Respiratory Synctial Virus, Human Parainfluenza), Reoviridae (Rotavirus), and Adenoviridae (Respiratory Adenovirus).

Disclosed herein are novel compositions and methods for alleviating and preventing symptoms associated with the common cold.

Disclosed herein are novel compositions and methods for alleviating and preventing symptoms associated with the common cold wherein such symptoms comprise rhinorrhea, runny nose, general congestion, nasal congestion, sneezing, fever, sore throat, cough, headache, body ache, muscle aches, muscle weakness, malaise, exhaustion, uncontrollable shivering, chills, otitis media, loss of appetite, pneumonia and bronchiolitis.

Disclosed herein are novel compositions and methods for alleviating and preventing symptoms associated with the common cold wherein such compositions comprise antiviral agents, including but not limited to, pleconaril.

Disclosed herein are novel compositions and methods for alleviating and preventing symptoms associated with the common cold wherein such compositions comprise antiviral agents, including but not limited to, pleconaril optionally combined with one or more pharmaceutical agents.

Disclosed herein are novel compositions and methods for alleviating and preventing symptoms associated with the common cold such as those caused by Picornaviridae infection wherein such compositions may be delivered in low dosages.

Disclosed herein are novel compositions and methods for alleviating and preventing symptoms associated with the common cold such as those caused by Picornaviridae infection wherein such compositions are optimized for ease of delivery, for dosing, for stability and reduced toxicity.

Disclosed herein are novel compositions and methods for alleviating and preventing symptoms associated with the common cold such as those caused by Picornaviridae infection wherein such compositions are optimized for ease of delivery to subjects having limited ability such as infants, elderly, immunocompromised individuals and subjects having restricted inspiratory airflow.

Disclosed herein are novel compositions and methods for alleviating and preventing symptoms associated with the common cold such as those caused by Picornaviridae infection wherein such compositions arc optimized for case of delivery to subjects, and wherein such compositions comprise pleconaril.

Disclosed herein are novel compositions and methods for alleviating and preventing symptoms associated with the common cold such as those caused by Picornaviridae infection wherein such methods facilitate and encourage therapeutic compliance.

Disclosed herein are novel compositions and methods for alleviating and preventing symptoms associated with the common cold and Picornaviridae infection wherein such symptoms comprise rhinorrhea, congestion, fever, sore throat, cough, headache, body ache, exhaustion, chills, otitis media, pneumonia and bronchiolitis and wherein such compositions are optimized for ease of delivery, for dosing, for stability and reduced toxicity, and wherein such compositions comprise compositions and methods comprising unique formulations of pleconaril.

Disclosed herein are novel compositions and methods comprising unique formulations of pleconaril and related formulations, combined with pharmaceutically acceptable excipients.

Disclosed herein are novel compositions and methods comprising unique formulations of pleconaril wherein such formulations are optimized for delivery to a subject via inhalation.

Disclosed herein are novel compositions and methods comprising unique formulations of pleconaril wherein such formulations are optimized for delivery to a subject via inhalation for site-specific delivery.

Disclosed herein are novel compositions and methods comprising unique formulations of pleconaril wherein such formulations are optimized for delivery to a subject via inhalation for site-specific delivery, wherein such sites comprise the nasopharynx and anterior internal pares.

Disclosed herein are novel compositions and methods comprising unique formulations of pleconaril wherein such formulations are optimized for delivery to a subject via inhalation.

These and other features and advantages of the present invention will become apparent after a review of the following detailed description of the disclosed embodiments and the appended claims.

### BRIEF DESCRIPTION OF THE FIGURES

FIG. 1 provides Table 1 showing treatment emergent adverse events for Study 843-203 (Example 1).

FIG. 2 provides log-transformed virus titers from serial nasal wash samples in Study 843-203 (Example 1).

FIG. 3 provides Table 2 showing data collected for Study 843-204: Summary of Mean (SD) Midazolam Pharmacokinetic Parameters for Days 1 and 14 and Geometric Mean Ratio of the Parameters and the 90% Confidence Limits (Example 2).

FIG. 4 provides Table 3 showing data collected for Study 843-204: Summary of Mean (SD) 1-OH-Midazolam Pharmacokinetic Parameters for Days 1 and 14 and Geometric Mean Ratio of the Parameters and the 90% Confidence Limits (Example 2).

### DETAILED DESCRIPTION

The present invention may be understood more readily by reference to the following detailed description of the specific embodiments included herein. Although the present invention has been described with reference to specific details of certain embodiments thereof, it is not intended that such details should be regarded as limitations upon the scope of the invention.

The entire text of the references mentioned herein are hereby incorporated in their entireties by reference including U.S. Provisional Patent Application Ser. No. 61/866,090, U.S. Pat. Nos. 5,464,848, 5,643,929, 7,429,606, 7,585,884, and United States Patent Application Publication Nos.: 20060167109, 20060229344, 20070202050, 20070203104, and 20090291990.

The present invention provides improved methods and compositions for alleviating and preventing symptoms associated with the common cold, particularly those viral infections and diseases associated with Picomaviridae (i.e. Enterovirus, Hepatovirus, Rhinovirus), Coronaviridae (SARS virus), Orthomyxoviridae (Influenzavirus) Paramyxovirinae (Pneumovirinae, Paramyxovirus, Respiratory Syncytial Virus, Human Parainfluenza), Reoviridae (Rotavirus), and Adenoviridae (Respiratory Adenovirus). The typical symptoms of the common cold include, but are not limited to, rhinorrhea, runny nose, general congestion, nasal congestion, sneezing, fever, sore throat, cough, headache, body ache, muscle aches, muscle weakness, malaise, exhaustion, uncontrollable shivering, chills, otitis media, loss of appetite, pneumonia and bronchiolitis.

More specifically, disclosed herein are formulations comprising optimized anti-viral agents having improve therapeutic efficacy. In some embodiments, efficacy is improved by altering therapeutic load, by modifying concentration, by adding one or more synergistic pharmaceutical agents, or by optimizing targeted delivery. In additional embodiments, the particles of the therapeutic agent may be altered to improve absorption, and/or to improve delivery dynamics. In addition, the compositions of the present invention are further desirable as they are designed to have a reduction in adverse events and drug-drug interactions (for example by eliminating first pass metabolism of the agents, especially in the cytochrome P450 (CYP), specifically CYP3A, superfamily in human liver and intestine). Additional improvements include expansion of the therapeutic window by controlling the dosing regimen and customizing therapeutic regimens to address the highly variable response seen in treating infants and other difficult to treat patient populations.

Pleconaril (3-3,5-dimethyl-4-[3-(3-methylisoxazol-5-yl)propoxy]phenyl-5-(trifluoromethyl) -1,2,4-oxadiazole) comprised a first of a new class of antiviral agents designed to treat infections caused by picornaviruses, the primary etiologic agents of the “common cold.” It was hypothesized that inhibition of picornavirus replication would reduce the severity and shorten the duration of cold symptoms. Pleconaril was rationally designed based on atomic resolution structures of drug/virus complexes, analyses of structure-activity relationships, screens for metabolic stability in liver microsomes, and extensive nonclinical safety testing. The pleconaril New Drug Application (NDA) submitted Jul. 31, 2001, was the result of sustained efforts to design and conduct successful clinical safety and efficacy trials.

Though not wishing to be bound by the following theory, it is thought that pleconaril inhibits picornavirus replication by direct interaction with the viral capsid and by inhibition of essential virus functions associated with the capsid. The picornavirus infection cycle begins with virus attachment to susceptible cells, followed by virus penetration into the cell. Once in the cell, the virus particle is disassembled, or uncoated, allowing for the release of viral RNA for subsequent viral protein production and RNA replication. Viral proteins and progeny RNA genomes then assemble into new virus particles. Finally, mature virions are released typically by destruction (lysis) of the infected cell. The picornavirus capsid is thought to be critically important in the virus attachment, uncoating, and maturation phases of the infection cycle.

The antiviral effects of pleconaril can be observed in the early stages of virus replication and upon maturation of progeny virions. Specifically, by interfering with the capsid, pleconaril prevents attachment of the majority of rhinoviruses to host cells and inhibits the uncoating of viral RNA of both rhinoviruses and enteroviruses. Further, when infected cells are exposed to pleconaril after the uncoating stage, the drug blocks the infectivity of progeny virions upon virus assembly.

Pleconaril exhibits a broad activity against human respiratory picornavirus as shown during clinical studies of an oral dosage form (ViroPharma NDA). However oral delivery of this medication at 200-400 mg dose level introduces systematic exposure and causing side effects including the induction of CYP 3A, which resulted in negative complications including not limited to, an increase in steady-state plasma concentration of theophylline, and increase incidence of menstrual irregularities in women on oral contraceptives.

Until now, delivery of pleconaril has also proved problematic. In its original format, delivery of pleconaril using a nasal thixotropic formulation (ViroPharma IND Report, 2004), demonstrated that a 12 mg per day (nasal spray) may produce roughly similar clinical/antiviral effect to 400 mg oral dose without any significant systemic exposure. Although a thixotropic formulation becomes fluid upon agitation, when shear is not applied to the system, the formulation viscosity increases, which is not conducive to routine pharmaceutical use. Unfortunately there are several challenges that limit successful development of pleconaril nasal delivery. Most importantly, pleconaril is practically insoluble in aqueous solutions (solubility in water <20 ng/ml at 25° C.) and therefore has both low equilibrium concentration and slow dissolution rate in relevant bio-spaces where picornavirus resides (predominantly, adenoids). The residence time of medication in the frontal turbinates and adenoids is relatively short for nasal sprays due to leakage and mucociliary clearance whereas spraying into the blocked nostril or nose with rhinorrhea may further reduce absorption and sneezing may also expel the medication. As a result of the above limitations, despite its therapeutic potential, effective delivery of pleconaril has remained elusive until now.

The inventors herein have overcome the problems (undesirable systemic exposure, negative side effects, poor delivery etc) associated with the original formulation of pleconaril by developing and implementing a drug design strategy that optimizes particle design, formulation and delivery. In one aspect, the inventors have enabled site specific delivery of pleconaril, controlled by size of particles delivered (potentially bi or tri-modal distribution of pleconaril particle from sub-micron range approximately 1-50, 5-40, 10-35 and 30+micron) or use of a novel delivery device to deliver to the nasopharynx and anterior internal pares, the locations where infection has the highest likelihood in resulting in an acute respiratory infection or cold. The present invention discloses drug delivery advantages for enhanced therapeutic activity including a significantly reduced size of pleconaril particles wherein 90% of the particles are less than 1 micron constituting nanosuspensions with enhanced dissolution properties, increased therapeutic activity and reduced drug dose but formulated in a manner that prevents these particles from lung delivery. More particularly, it has been discovered herein that administration of such suspensions allows for lower dosage levels than would be necessary to achieve a similar therapeutic response by other methods of delivery (e.g. nasal micro-particulate delivery) for reduction of systemic side effects. The dosing regimen disclosed herein comprises approximately 1-80 mg, 10-50 mg, 10-40 mg once or twice daily. In addition to increased therapeutic efficacy, such nanosuspensions also offer enhanced therapeutic efficiency.

Another aspect of this invention is that nanosuspensions are delivered in enhanced formulations for lower clearance of medication from relevant bio-spaces, maximizing nasal drug distribution and providing enhanced droplet dispersion of the nasal sprays when compared to micro-formulations in the prior art. In certain embodiments, the pleconaril formulation may comprise thixotropic agents. Surprisingly, this formulation method produces a greater stability of nanosuspensions compared to microsuspensions.

In an alternative embodiment, pleconaril is formulated as an in situ gel thereby increasing local pleconaril residence, slowing systemic exposure and obtaining unexpected sustained action. In this embodiment, the gel may be applied to the nose and nasal for sustained delivery. In the gel format also, particle size may be controlled for optimal results.

Additional aspects of the improved formulations described herein include combination of pleconaril with one or more excipients. Certain excipients may provide both mucoadhesive actions to prolong the residence time in the bio-spaces. Other excipients may possess synergetic antiviral properties enhancing the therapeutic effect of pleconaril: co-administration allows delivery of both agents to the same region of bio-spaces, creating a microenvironment where pleconaril activity is increased. For example, some formulations include pleconaril combined with other antiviral agents, for example effective treatments for respiratory syncytial virus (RSV) or influenza, i.e. oseltamivir phosphate. Yet another aspect of this invention is preparation of pleconaril formulation in the form of solid composite microparticles whereby the drug is blended into uniform solid phase with selected excipients in order to increase the drug dissolution rate, to provide mucoadhesive properties after the delivery of microparticles into bio-spaces, and/or combine antiviral properties of excipients synergistically to provide a more significant and/or prolonged therapeutic antiviral effect greater than that achieved by the pleconaril alone.

Disclosed herein are further improvements and enhancements including, but not limited to inclusion complexes. Inclusion complexes generally fall within the category of host-guest chemistry and comprise the insertion of the drug, into the cavity of another molecule or group of molecules. In some embodiments, such inclusion complexes comprise a solid solution in which molecules of one compound occupy places in the crystal lattice of another compound. Inclusion complexes include cyclodextrins. Disclosed herein are inclusion complexes comprising cyclodextrins and pleconaril. Disclosed herein are inclusion complexes comprising cyclodextrins and pleconaril having improved microparticle structuring to optimize therapeutic delivery and efficacy.

Disclosed herein are further improvements and enhancements including, but not limited to embodiments wherein pleconaril is incorporated into micelles formed with surfactants, including nonionic surfactants include polysorbates, polyoxyethylated castor oil. Also disclosed are improved pleconaril formulations further comprising oil, polyoxyethylated glycerides, lauroyl macroglycerides, and mono- and di-fatty acid esters of low molecular weight polyethylene glycols.

Disclosed herein are further improvements and enhancements including, but not limited to embodiments wherein pleconaril is formulated as a nanosuspension and wherein the particle size of pleconaril is less than 1 micrometer, between 100-800 nanometers, between 200 and 700 nanometers, between 300 and 700 nanometers and approximately 500 nanometers.

Disclosed herein are further improvements and enhancements including, but not limited to embodiments wherein the pleconaril formulation is biocompatable, biodegradable and bioadhesive. Such embodiments include, but are not limited to unique pleconaril formulations comprising cross-linked starch microspheres, for optimized delivery and retention in the nasal cavity where rapid mucociliary clearance limits drug contact with mucosal membranes.

Disclosed herein are further improvements and enhancements including, but not limited to embodiments wherein the pleconaril formulation comprises pleconaril suspensions with thixotropic properties by carefully controlling, temperature, polymer concentration, polymer combinations and addition of cations or certain excipients.

Disclosed herein are further improvements and enhancements including, but not limited to embodiments wherein the pleconaril formulation comprises pleconaril or a pharmaceutically acceptable salt thereof, wherein said solution comprises at least one solvent selected from the group consisting of pleconaril-dissolving pharmaceutically acceptable oils, hydrofluorocarbons, and mixtures of two or more thereof.

Disclosed herein are further improvements and enhancements including, but not limited to embodiments wherein the pleconaril formulation comprises pleconaril or a pharmaceutically acceptable salt thereof, wherein said solution comprises one or more solvents selected from the group consisting of ester mixtures or mixture of saturated fatty acids.

Disclosed herein are further improvements and enhancements including, but not limited to embodiments wherein the pleconaril formulation comprises a solution employing pleconaril-dissolving hydrofluorocarbons wherein said formulation is suitable for administration from a pressurized metered dose inhaler device.

Disclosed herein are further improvements and enhancements including, but not limited to embodiments wherein the pleconaril formulation comprises a nasal spray with optimized droplet particle size to effectively deliver to, and cover a bio-target in the nasal cavity for pleconaril. In certain embodiments, for example preferred dispersion comprises droplets having an average diameter of from about 10 microns to about 120 microns, and wherein 90% of the droplets have a diameter of not more than 220 microns.

Disclosed herein are further improvements and enhancements including, but not limited to embodiments wherein the pleconaril formulation is provided in a dosage of approximately 1 mg to about 600 mg, 10 to 400 mg, 10 to 200 mg, or 10 to 40 mg in single or divided doses daily for a period sufficient to treat a condition, for example, a viral infection, or more particularly, a viral induced respiratory infection.

Disclosed herein are medicaments utilizing a solution containing pleconaril that is incorporated into any other dosage form suitable for incorporation of a liquid and delivering via alternate routes of administration, including oral, parenteral and transdermal.

Further optimization of the formulations of the present invention comprise identifying effective dosing strategies: customize drug dose by patient population, number of doses per day, number of treatment days based on the correlation/benefit of reduction in viral shedding and or symptom reduction.

The improved compositions and formulations disclosed herein include methods of treating acute respiratory wheezing illness in hospitalized children, including bronchiolitis and acute asthma) by treating RSV, enterovirus and rhinovirus infections, methods of treating asthma exacerbations by treating the presence and replication of rhinovirus in the lower airways, methods of treating myocarditis due to Coxsackievirus B and methods of treating febrile wheeze due to HRV-C Glade. In further embodiments, the present invention comprises the optimization of anti-viral formulations to include combinations with anti-viral therapies for RSV, bronchodilators, antibiotics and/or anti-fungals.

It should be emphasized that the above-described embodiments of the present invention and process, particularly, and “preferred” embodiments, are merely possible examples of implementations and merely set forth for a clear understanding of the principles of the disclosure. All these and other such modifications and variations are intended to be included herein within the scope of this disclosure and protected by the following claims. Therefore the scope of the disclosure is not intended to be limited except as indicated in the appended claims.

In general, when referring to treatment, the therapeutic compositions discussed herein may be administered orally, parenterally (e.g., intravenously or subcutaneous administration), by intramuscular injection, by intraperitoneal injection, transdermally, extracorporeally, by intracavity administration, transdermally, or topically or the like, including topical intranasal administration or administration by inhalant. The topical administration can be ophthalmically, vaginally, rectally, or intranasally. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

As used herein, parenteral administration” of the composition, if used, is generally characterized by injection. Injectables can be prepared in conventional forms, either as liquid solutions or suspensions, solid forms suitable for solution of suspension in liquid prior to injection, or as emulsions. Parenteral administration includes use of a slow release, a time release or a sustained release system such that a constant dosage is maintained.

The term “therapeutically effective” means that the amount of the composition used is of sufficient quantity to ameliorate one or more causes or symptoms of a disease or disorder, such as aberrant cell growth, tumor development, and cancer. Such amelioration only requires a reduction or alteration, not necessarily elimination. Effective dosages and schedules for administering the disclosed compositions may be determined empirically, and making such determinations is within the skill in the art. The dosage ranges for the administration of the compositions are those large enough to produce the desired effect in which the symptoms of the disorder arc affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

The specific effective amount of a therapeutic for any particular subject or patient will depend upon a variety of factors including the disease or disorder being treated and the severity of the disorder; the identity and activity of the specific composition employed; the age, body weight, general health, sex and diet of the patient; the time of administration; the route of administration; the rate of excretion of the specific composition employed; the duration of the treatment; drugs used in combination or coincidental with the specific composition employed and like factors well known in the medical arts.

For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of ischemia-reperfusion injury, trauma, drug/toxicant induced injury, neurodegenerative disease, cancer, or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular subject or patient: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious.

The effective amount of a prescribed therapeutic may be given daily, every other day, weekly, monthly, bi-monthly, every other monthly, yearly, or at any other interval that is determined by the physician or provider to be effective. For example, the effective daily dose can be divided into multiple doses for purposes of administration. Consequently, single dose therapeutic can contain such amounts or submultiples thereof to make up the daily dose. Disclosed therapeutics can also be administered as part of a combination of anti-tumor or anti-cancer treatments. In an aspect, disclosed compositions can be administered to the subject or patient prior to treatment with an anti-tumor or anti-cancer treatment. In an aspect, disclosed compositions can be administered concurrently with the anti-tumor or anti-cancer treatment. In an aspect, disclosed composition can be administered subsequent to the anti tumor or anti-cancer treatment. In an aspect, the patient or subject receives both treatments on an alternating or rotating schedule. In an aspect, the subject or patient receives a singular treatment with the disclosed composition. In an aspect, the subject or patient receives at least one treatment with the disclosed composition. In an aspect, the subject or patient receives at least one treatment with the disclosed composition and at least one other anti-tumor or anti cancer treatment.

The dosage can be adjusted by the individual physician or the subject in the event of 20 any counter-indications. Dosage can vary, and can be administered in one or more dose administrations daily, for one or several days. Guidance can be found in the literature for appropriate dosages for given classes of pharmaceutical products.

The terminology used herein is for the purpose of describing particular aspects only and is not intended to be limiting.

As used in the specification and the appended claims, the singular forms “a,” “an” and “the” can include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%. When such a range is expressed, an aspect includes from the one particular value and/or to the other particular value. Similarly, when values are expressed as approximations, by use of the antecedent “about,” it will be understood that the particular value forms an aspect. It will be further understood that the endpoints of each of the ranges are significant both in relation to the other endpoint, and independently of the other endpoint.

The word “or” as used herein means any one member of a particular list and also includes any combination of members of that list.

“Inhibit,” “inhibiting,” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100 percent, or any amount of reduction in between as compared to native or control levels. In an aspect, the inhibition or reduction is 10-20, 20-30, 30-40, 40-50, 50-60, 60-70, 70-80, 80-90, or 90-100 percent as compared to native or control levels. In an aspect, the inhibition or reduction is 0-25, 25-50, 50-75, or 75 100 percent as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100 percent, or more, or any amount of promotion in between compared to native or control levels. In an aspect, the increase or promotion is 10-20, 20-30, 30-40, 40-50, 50-60, 60-70, 70-80, 80-90, or 90-100 percent as compared to native or control levels. In an aspect, the increase or promotion is 0-25, 25-50, 50-75, or 75-100 percent, or more, such as 200, 300, 500, or 1000 percent more as compared to native or control levels. In an aspect, the increase or promotion can be greater than 100 percent as compared to native or control levels, such as 100, 150, 200, 250, 300, 350, 400, 450, 500 percent or more as compared to the native or control levels.

As used herein, the term “determining” can refer to measuring or ascertaining a quantity or an amount or a change in activity. For example, determining the amount of a disclosed polypeptide in a sample as used herein can refer to the steps that the skilled person would take to measure or ascertain some quantifiable value of the polypeptide in the sample. The art is familiar with the ways to measure an amount of the disclosed polypeptides and disclosed nucleotides in a sample.

The term “sample” can refer to a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g., a polypeptide or nucleic acid). A sample may also be any body fluid or excretion (for example, but not limited to, blood, urine, stool, saliva, tears, bile) that contains cells or cell components.

The invention will be further described with reference to the following examples; however, it is to be understood that the invention is not limited to such examples. Rather, in view of the present disclosure that describes the current best mode for practicing the invention, many modifications and variations would present themselves to those of skill in the art without departing from the scope and spirit of this invention. All changes, modifications, and variations coming within the meaning and range of equivalency of the claims are to be considered within their scope.

### EXAMPLES

### Example 1

Efficacy and Safety of Pleconaril Nasal Spray on Experimentally Induced Human Rhinovirus Respiratory Infection in Healthy Adults (Study 843-203)

A nasal inoculation study (using RV-39) was performed on 93 healthy volunteers treated with pleconaril nasal spray BID, pleconaril nasal spray TID, or oral pleconaril for 5 days. This study demonstrated pleconaril was effective in reducing common cold symptom duration and viral titers. No serious adverse events were reported in this study. It was also observed that the scrum concentration of intranasal plcconaril was greater than 100-fold lower than the oral administration of pleconaril.

In this randomized, double-blind, placebo-controlled study, 93 healthy adult subjects received pleconaril nasal spray at one of two dose levels (BID: Day 1=9 mg/day and Days 2&3=6 mg/day or TID: Day 1=12 mg/day and Days 2&3=9 mg/day), vehicle nasal spray, pleconaril oral tablets (400 mg TID×5 days), or placebo tablets. Subjects were confined to a clinical facility with on-site medical surveillance from Day-2 through Day 5; outpatient follow-up visits were conducted on Days 5-7. On Day-1, 24 hours before administration of the first dose of study drug, each subject received an intranasal inoculation of 100-300 tissue culture infective dose 50% (TCTD50) rhinovirus type 39. Nasal wash specimens were collected pre-inoculation and once or twice daily at predetermined time points on Days 1-7 for virologic determinations. Nasal mucus weights were determined daily through Day 4. The presence and severity of clinical symptoms were assessed throughout the study period. Pleconaril administered as a nasal spray in a BID regimen (dosing up to 9 mg/day) and a TID regimen (dosing up to 12 mg/day) was well tolerated. There were no serious adverse events and no discontinuations of study drug due to adverse events. All adverse events reported in the pleconaril nasal spray groups were of mild or moderate intensity. One subject in the pleconaril BID nasal spray group had study drug interrupted temporarily due to “blood in nasal mucus” observed on Day 1; this event was considered mild and resolved without treatment, and the subject completed BID dosing on Days 2 and 3.

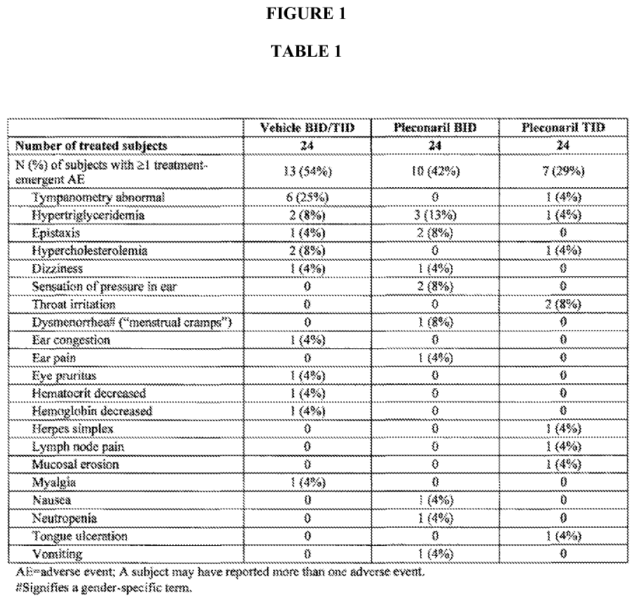

Four subjects (1 receiving vehicle nasal spray, 2 receiving pleconaril BID nasal spray, and 1 receiving pleconaril oral tablet) had events of epistaxis (reported as “blood in mucus” or “nosebleed”), all of which were mild and resolved without treatment. One subject receiving pleconaril TID nasal spray had a mild mucosal erosion (“2 mm erosion, left septum”) observed during rhinoscopic examination-13 hours after the last dose of study drug; there was no associated bleeding and this also resolved without treatment. Given the mild nature of these findings and the overall distribution of adverse events reported for subjects across the different study groups (i.e., pleconaril nasal spray, vehicle, or oral tablets), the potential for local irritation caused by pleconaril nasal spray was minimal. A summary of treatment-emergent adverse events is presented for subjects in the nasal spray groups in Table I (FIG. 1).

Analyses of clinical laboratory values and vital signs were unremarkable. Particular attention was paid to monitoring of pulmonary function, and no subject had any symptoms or signs suggestive of lower respiratory illness or bronchospasm. The proportion of subjects with reductions in PEFR of either greater than or equal to 20% or greater than or equal to 50 L/min were similar across the vehicle and pleconaril nasal spray groups (38% in vehicle nasal spray BID/TID groups and 25% and 33% in pleconaril nasal spray BID and TID groups, respectively). One subject (#00003) receiving pleconaril BID nasal spray had reductions in PEFR greater than or equal to 20% at 12 of 13 post-baseline time points, but this subject had no symptoms or signs that required medical intervention and the finding was not considered by the investigator as an adverse event.

Antiviral Activity

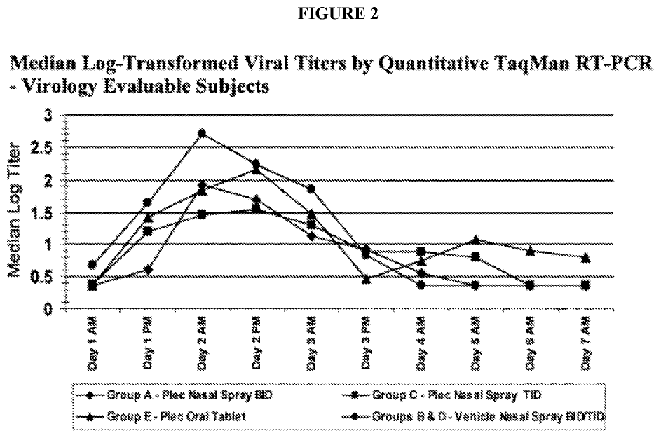

Primary Endpoint: Log-transformed virus titers from serial nasal wash samples in Study 843-203 are shown in FIG. 2 for virology evaluable subjects (i.e., those subjects with a pre-inoculation serum neutralizing antibody titer to rhinovirus type 39-1:2 and reverse transcriptase polymerase chain reaction [RT-PCR] and culture negative nasal wash sample and at least one post baseline nasal wash sample that was RT-PCR or culture positive). At the time of maximum viral titers (Day 2 AM) in the combined vehicle groups, median viral titers in each pleconaril group were 0.78-1.26 log lower than vehicle groups, and viral titers in the combined pleconaril BID/TID groups were 1 log lower than the vehicle groups. The repeated measures analysis of Day 1 PM to Day 7 was not significant for any between-group comparison. However, median log-transformed viral titers in each pleconaril treatment group (intranasal and oral) were lower than the vehicle groups at all measured time points from the time of initiation of study drug through Day 3 AM.

### Example 2

Effect of Pleconaril Nasal Spray on CYP 3A4 (Study 843-204)

Study 843-204 demonstrated that the low concentrations of pleconaril achieved after intranasal administration do not induce CYP 3A4. Study 843-204 was a double-blind, saline-controlled study conducted to evaluate the effect of repeat doses of plcconaril nasal spray on the pharmacokinetics of oral midazolam.

The data convincingly demonstrate that the small amount of pleconaril entering the systemic circulation after intranasal administration does not result in induction of CYP 3A4 as demonstrated by the unchanged exposure to oral midazolam, a sensitive probe of CYP 3A4 activity.

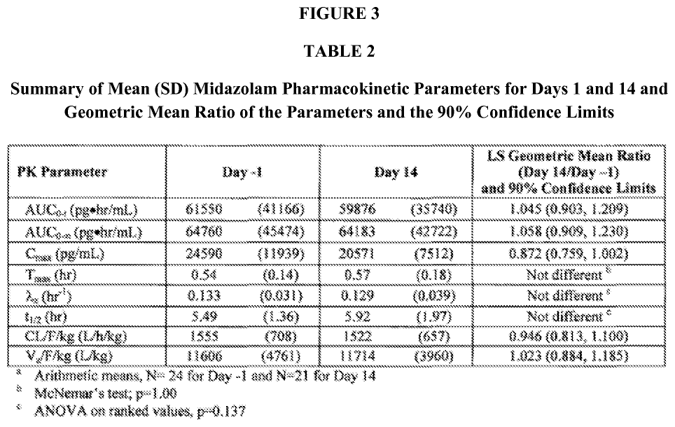

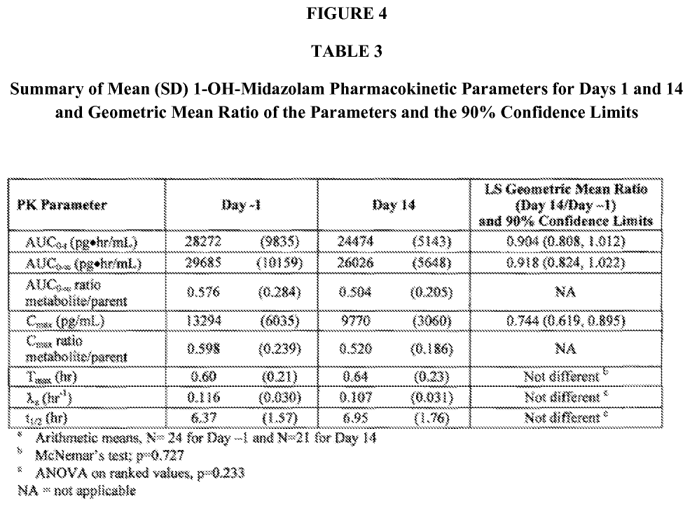

During the development program of oral pleconaril it was determined that pleconaril is an inducer of CYP 3A4. As part of the early development program of intranasal pleconaril, a PK interaction study was conducted to determine if the low systemic exposure to pleconaril observed when administered by the intranasal route was associated with changes in cytochromic P450 3A4 activity. Study 843-204 was a double-blind, saline-controlled study conducted to evaluate the effect of repeat doses of pleconaril nasal spray on the pharmacokinetics of oral midazolam. Thirty subjects (24 active treatment and 6 saline controlled subjects) were enrolled into the study. Subjects in the active treatment arm received intranasal pleconaril 3 mg TID for 40 doses over 14 days. Prior to dosing and on the final day of the study, subjects received oral midazolam 0.075 mg/kg. Plasma samples were obtained up to 22 hours postdose for determination of plasma midazolam and 1-hydroxymidazolam pharmacokinetics. Data from the subjects who received active drug were statistically analyzed and the 90% confidence intervals for log-transformed Cₘₐₓ and AUC were determined. The pharmacokinetic data for midazolam and 1-OH midazolam and the statistical analyses from the intranasal study are summarized in Table 2 (FIG. 3) and Table 3 (FIG. 4).

Following administration of pleconaril nasal spray BID (Day 1=9 mg/day and Days 2 and 3=6 mg/day or TID (Day 1=12 mg/day and Days 2 and 3=9 mg/day), plasma pleconaril was detectable in some samples but present at very low concentrations. This study demonstrated that plcconaril was effective in reducing common cold symptom duration and viral titers. No serious adverse events were reported in this study. It was also observed that the serum concentration of intranasal pleconaril was greater than 100-fold lower than the oral administration of pleconaril. The nasal and oral pleconaril groups showed significantly lower nasal mucus weights than the combined vehicle controls. The nasal and oral pleconaril groups also showed benefit vs. vehicle through day 3 post infection.

The data convincingly demonstrate that the small amount of pleconaril entering the systemic circulation after intranasal administration does not result in induction of CYP 3A4 as demonstrated by the unchanged exposure to oral midazolam, a sensitive probe of CYP 3A4 activity.

This single-sequence, repeat-dose, drug-interaction study was conducted to determine the effect of plcconaril nasal spray 3 mg TID over 14 days (40 doses) on the disposition of midazolam (a CYP 3A substrate) and the midazolam metabolite, 1-0H-midazolam in healthy adults. Following nasal spray treatment, pleconaril plasma concentrations were very low in all subjects. The highest observed pleconaril plasma concentration for any subject was 28 ng/mL. With respect to CYP 3A activity, the results of the study indicated that, with the exception of Cmax, the 90% confidence limits of the ratio of geometric means for both midazolam (AUC, Cl/F and Vz/F) and 1-0H-midazolam (AUC) pharmacokinetic parameters were contained within the interval (0.80,1.25). The exception was Cₘₐₓ for which midazolam and 1-OHmidazolam concentrations were slightly lower on Day 14, which might have resulted from a different rate of midazolam absorption on Day 14 than on Day 1. Pleconaril treatment had no effect on the elimination rate constant of midazolam or 1-0H-midazolam. In addition, pleconaril treatment was associated with lower plasma concentrations of 1-OH-midazolam and lower ratios of 1-OH-midazolam to midazolam for C., and AUC on Day 14; the opposite result would be expected for a CYP 3A inducer. The overall data set indicates no induction of CYP 3A activity following 14 days of intranasal pleconaril administration (3 mg TID [40 doses]). A summary of the pharmacokinetic results for midazolam and 1-OH-midazolam are presented below in Table 2 and Table 3, respectively.

What is claimed:

1. A composition comprising an antiviral therapeutic agent wherein said antiviral therapeutic agent comprises pleconaril,
wherein said pleconaril is optimized for therapeutic efficacy,
wherein said composition consists of pleconaril in an inclusion complex,
or wherein the pleconaril is incorporated into micelles,
and wherein the pleconaril particles in the inclusion complex
or in the micelles are in the size range of 1-50, 5-40, 10-35 microns.

2. The composition of claim 1, wherein said pleconaril is optimized for ease of delivery, for dosing, for stability and reduced toxicity.

3. The composition of claim 1, wherein said pleconaril is optimized for site-specific delivery, wherein such sites comprise the nasopharynx and anterior internal nares.

4. The composition of claim 1, wherein said pleconaril is designed to have a reduction in adverse events and drug-drug interactions.

5. The composition of claim 1, wherein the inclusion complex comprises cyclodextrins, and wherein the micelles are formed within surfactants.

6. The composition of claim 1, wherein the composition comprises a biocompatable, biodegradable and bioadhesive formulation.

7. The composition of claim 1, further comprising pharmaceutically acceptable oils, pharmaceutically acceptable excipients, or hydrofluorocarbons.

8. The composition of claim 7, wherein the composition is suitable for administration from a pressurized metered dose inhaler device.

9. The composition of claim 1, further comprising ester mixtures or mixture of saturated fatty acids.

10. The composition of claim 1, wherein the composition is administered to a subject in need thereof intranasally, orally, parenterally, intravenously, subcutaneously, intramusculary, intraperitoneally, transdermally, extracorporeally, by intracavity administration, transdermally, topically, by topical intranasal administration, by inhalation, or ophthalmically.

11. The composition of claim 1, wherein the composition is suitable for treating viral infections and diseases associated with Picornaviridae, Coronaviridae, Orthomyxoviridae, Paramyxovirinae, Reoviridae, and Adenoviridae.

12. A method for treating viral infections and diseases associated with Picornaviridae, Coronaviridae, Orthomyxoviridae, Paramyxovirinae, Reoviridae, and Adenoviridae comprising administering a composition comprising an antiviral therapeutic agent, wherein said antiviral therapeutic agent comprises pleconaril,
wherein said pleconaril is optimized for therapeutic efficacy,
wherein said composition consists of pleconaril in an inclusion complex, or wherein the pleconaril is incorporated into micelles,
and wherein the pleconaril particles in the inclusion complex or in the micelles are in the size range of 1-50, 5-40, 10-35 microns
and wherein administration of the pleconaril reduces symptoms associated with the viral infection.

13. The method of claim 12, wherein the composition is optimized for ease of delivery, for dosing, for stability and reduced toxicity.

14. The method of claim 13, wherein the composition is optimized for site-specific delivery, wherein such sites comprise the nasopharynx and anterior internal nares.

15. The method of claim 12, wherein the symptoms associated with the viral infection comprise rhinorrhea, runny nose, general congestion, nasal congestion, sneezing, fever, sore throat, cough, headache, body ache, muscle aches, muscle weakness, malaise, exhaustion, uncontrollable shivering, chills, otitis media, loss of appetite, pneumonia and bronchiolitis.

16. The method of claim 12, wherein the composition further comprises pharmaceutically acceptable oils, pharmaceutically acceptable excipients, or hydrofluorocarbons.

17. The method of claim 12, wherein the composition is administered to a subject in need thereof intranasally, orally, parenterally, intravenously, subcutaneously, intramusculary, intraperitoneally, transdermally, extracorporeally, by intracavity administration, transdermally, topically, by topical intranasal administration, by inhalation, or ophthalmically.

18. The method of claim 12, wherein the composition comprises a biocompatible, biodegradable and bioadhesive formulation.

19. The method of claim 12, wherein the composition is suitable for administration from a pressurized metered dose inhaler device.

20. The method of claim 12, wherein the composition further comprises ester mixtures or a mixture of saturated fatty acids.
